(19) United States
(12) Patent Application Publication
(10) Pub. No.: US 2010/0048594 A1
(43) Pub. Date: Feb. 25, 2010

(54) USE OF CYTOHESIN INHIBITORS FOR CHEMICALLY INDUCING LONGEVITY

(75) Inventors: Michael Famulok, Bonn (DE); Markus Hafner, Bonn (DE); Anton Schmitz, Bonn (DE); Bernhard Fuss, Bonn (DE); Ingo Zinke, Siegburg (DE); Michael Hoch, Swisttal (DE)

Correspondence Address: RANKIN, HILL & CLARK LLP, 38210 Glenn Avenue, WILLOUGHBY, OH, 44094-7808, US

(73) Assignee: RHEINISCHE FRIEDRICH-WILHELMS UNIVERSITAT, Bonn (DE)

(21) Appl. No.: 12/514,861

(22) PCT Filed: Nov. 14, 2007
(86) PCT No.: PCT/EP07/62337

§ 371 Date: Jul. 16, 2009

(30) Foreign Application Priority Data
Nov. 15, 2006 (DE) 10 2006 054 205.3

(51) Int. Cl. A61K 31/4196; A61K 31/505; A61K 31/341; A61K 31/343; A61P 1/16; A61P 3/06; A61P 1/18

(52) U.S. Cl. 514/274; 514/384; 514/461; 514/466

(57) ABSTRACT

The invention relates to compounds selected from among the group comprising general formulas (1), (2), (3), and/or (4) and/or the enantiomers, diastereomers, and derivatives thereof, and the pharmaceutically acceptable salts thereof for producing a medicament used for therapeutically and/or preventively treating disease and pathological conditions linked to regulation of the insulin and/or insulin-like growth factor (IGF) signalling pathway and/or for chemically inducing longevity.

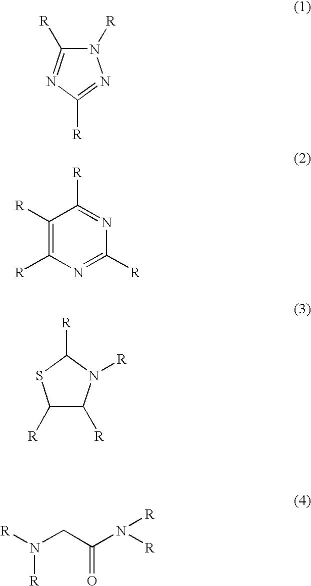

The invention relates to the use of compounds of the general formulas (1), (2), (3) and (4) for treating and/or preventing diseases and pathological conditions that are linked to a regulation of the insulin and/or insulin-like growth factor (IGF) signaling pathway, and/or for chemically inducing longevity.

Compounds of the general formulas (1), (2), (3), and (4) are known from the publication DE 10 2004 055 998 A1.

Every cell function, including cell aging and cell death, is controlled by a multitude of cell signaling pathways. The development of some diseases is also dependent upon cell aging, and the probability of dying of diseases increases with age.

Another widespread problem connected with today's lifestyle is obesity, also called adiposity. This is a key factor in the development of many chronic illnesses as well as some types of cancer.

The object of the present invention was to provide compounds that are capable of overcoming at least one of the disadvantages of the prior art. Especially, the object was to provide means that will allow the insulin signalling pathway to be influenced.

This object is attained through the use of compounds selected from the group comprising the general formulas (1), (2), (3) and/or (4) as indicated below:

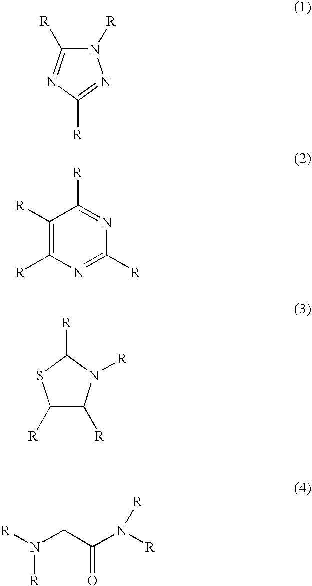

wherein:
R is selected, the same or each independently of the others, from the group comprising hydrogen, OH, COOH, COO(C₁-C₁₀-alkyl), CONH₂, CONH(C₁-C₁₀-alkyl), CON(C₁-C₁₀-alkyl)₂, NHCO(C₁-C₁₀-alkyl), NHCOCHCl₂, halogen, preferably selected from the group comprising Cl, Br, F, CF₃, amine, C₁-C₁₀-alkyl, C₁-C₁₀-alkoxy and/or a structural element (A1), (B1), (C1), (D1), (E1), (F1), (G1), (H1), (I1), (J1), (L1), (M1) as indicated below:

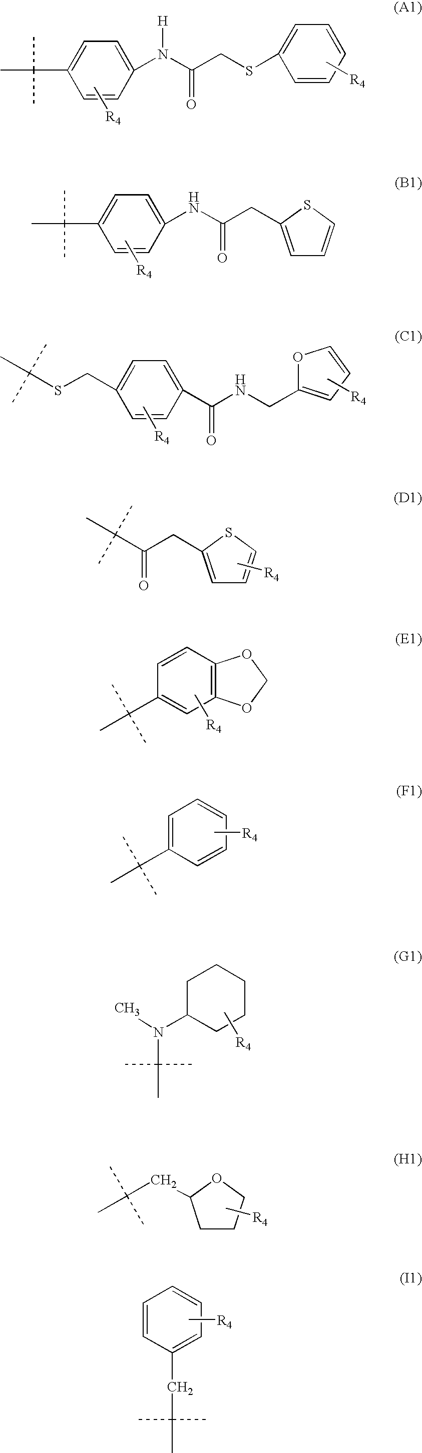

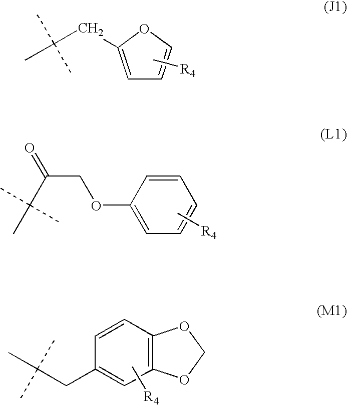

wherein:
R₄ is selected, the same or each independently of the others, from the group comprising hydrogen, OH, COOH, COO(C₁-C₁₀-alkyl), CONH₂ CONH(C₁-C₁₀-alkyl), CON(C₁-C₁₀-alkyl)₂, NHCO(C₁-C₁₀-alkyl), NHCOCHCl₂, halogen, preferably selected from the group comprising Cl, Br, F, CF₃, amine, C₁-C₁₀-alkyl and/or C₁-C₁₀-alkoxy,
   and/or the enantiomers, diastereomers, derivatives and pharmaceutically well-tolerated salts thereof, for the production of a pharmaceutical preparation for the therapeutic and/or preventive treatment of diseases and pathological conditions that are linked to a regulation of the insulin and/or insulin-like growth factor (IGF) signaling pathway and/or for chemically inducing longevity.

It was surprisingly found that the compounds that can be used according to the invention are able to influence the insulin and/or insulin-like growth factor (IGF) signaling pathway. It was especially surprisingly found that the compounds that can be used according to the invention can lead to an insulin resistance, i.e., decreased activity of the insulin signaling pathway, in mice.

Without being committed to a given theory, it is assumed that the influencing of the insulin and/or insulin-like growth factor (IGF) signaling pathway and the insulin resistance are based upon an inhibition of the cytohesins, especially of cytohesin-1, cytohesin-2, cytohesin-3 and/or cytohesin-4, by the compounds that can be used according to the invention.

It was surprisingly found that compounds selected from the group comprising the general formulas (1), (2), (3) and/or (4) can lead to chemically induced longevity.

It was further surprisingly found that the compound according to formula (9) can lead to significantly improved chemically induced longevity, for example to a significant increase in the lifespan of flies. It is especially advantageous that especially the compound according to formula (9) can induce an activation of the immune system.

Preferably, the compounds that can be used according to the invention have at least one, preferably two, especially preferably three of the same and/or different structural elements (A1), (B1), (C1), (D1) and/or (E1). In other preferred embodiments, the compounds that can be used according to the invention have at least one structural element selected from the group comprising (E1), (F1), (G1), (H1), (I1), (J1), (K1), (L1), (M1) and/or (O1).

In preferred embodiments, the structural element R₄ is selected, the same or each independently of the others, from the group comprising hydrogen, NHCOCHCl₂, Cl, CF₃ and/or F.

One particular advantage of the compounds that can be used according to the invention is that the compounds can especially also be used in preventive applications. This makes it possible to use the compounds that can be used according to the invention not only to treat existing pathological conditions, but also for preventive applications, for example to prevent age-related cell damage or obesity.

The term “preventive treatment” within the context of the present invention especially means that the compounds that can be used according to the invention can be administered preventively, for example before age-related cell damage occurs. Of particular advantage is the fact that, for example, obesity can be avoided through preventive treatment.

In preferred embodiments, the compounds are selected from the group comprising the general formulas (1), (2), (3) and/or (4) selected from the group comprising compounds of the general formulas (5), (6), (7) and/or (8) as indicated below and/or the enantiomers, diastereomers, derivatives and pharmaceutically well-tolerated salts thereof:

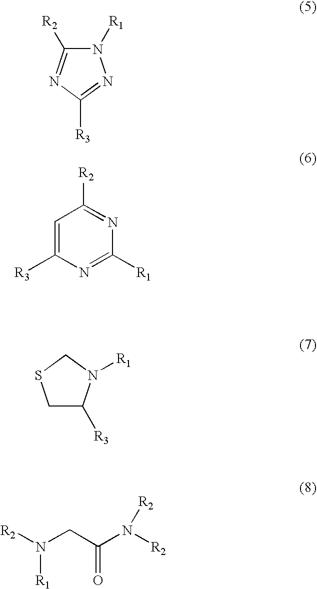

wherein:
R_{1′} is selected, the same or each independently of the others, from the group comprising R₂, R₃ and/or a structural element (A2), (B2), (C2), (D2), (N2) as indicated below:

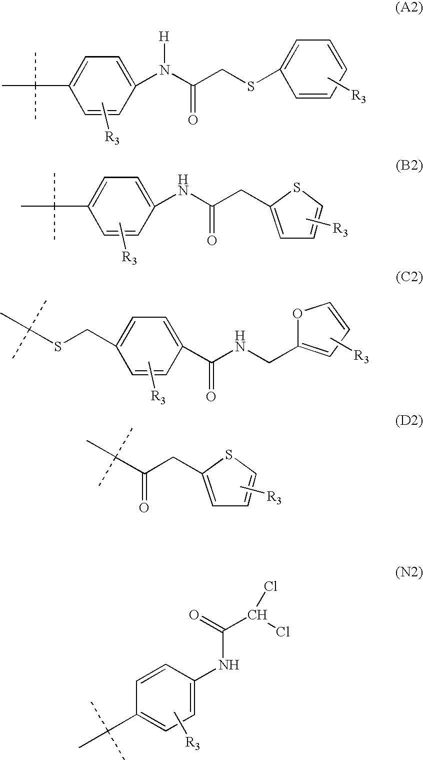

R₂ is selected, the same or each independently of the others, from the group comprising R₁, R₃ and/or a structural element (E2), (F2), (G2), (H2), (12), (J2), (K2), (L2), (M2), (O2) as indicated below:

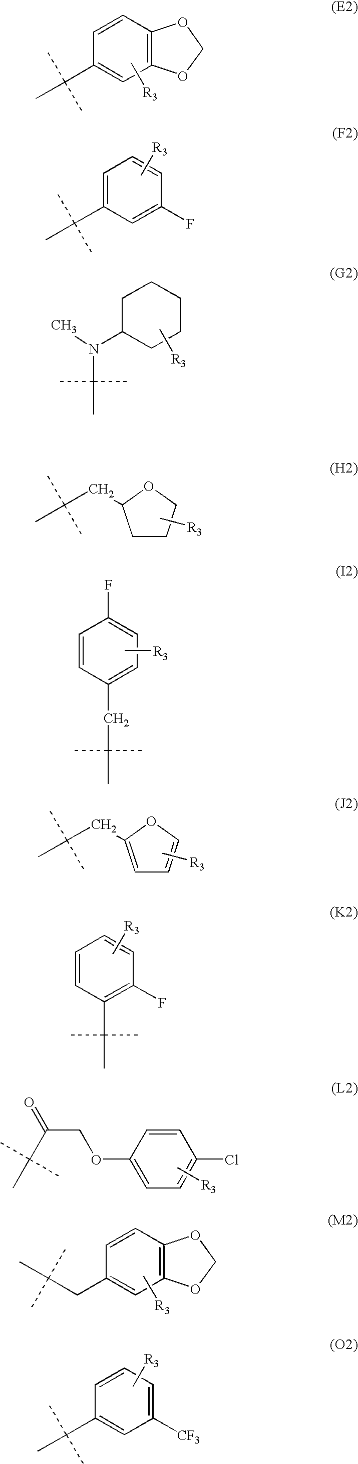

R₃ is selected, the same or each independently of the others, from the group comprising R₁, R₂ and/or selected from the group comprising hydrogen, OH, COOH, COO(C₁-C₁₀-alkyl), CONH₂, CONH(C₁-C₁₀-alkyl), CON(C₁-C₁₀-alkyl)₂, NHCO(C₁-C₁₀-alkyl), NHCOCHCl₂, halogen, preferably selected from the group comprising Cl, Br, F, CF₃, amine, C₁-C₁₀-alkyl and/or C₁-C₁₀-alkoxy.

Preferably, at least one or more of the structural elements R₁, R₂ and/or R₃ are selected, the same or each independently of the others, from the sulfurous structural elements (A2), (B2), (C2) and/or (D2). The compounds that can be used according to the invention can have several of the same and/or different structural elements (A2), (B2), (C2) and/or (D2). Preferably, the compounds that can be used according to the invention have at least one, preferably two, especially preferably three of the same and/or different structural elements (A2), (B2), (C2) and/or (D2). Also preferably, the compounds that can be used according to the invention have at least one structural element selected from the group comprising (E2), (F2), (G2), (H2), (12), (J2), (K2), (L2), (M2) and/or (O2). Preferably, the structural element R₃ of the structural elements (E2), (F2), (G2), (H2), (12), (J2), (K2), (L2), (M2) and/or (O2) is hydrogen.

In further preferred embodiments, the compounds that can be used according to the invention are selected from the group comprising compounds of the general formulas (5), (6), (7) and/or (8) as indicated below, and/or the enantiomers, diastereomers, derivatives and pharmaceutically well-tolerated salts thereof:

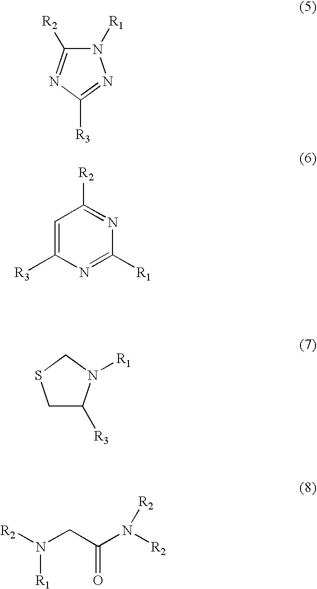

wherein
R₁ is selected, the same or each independently of the others, from the group comprising R₂, R₃ and/or a structural element (A3), (B3), (C3), (D3), (N3) as indicated below:

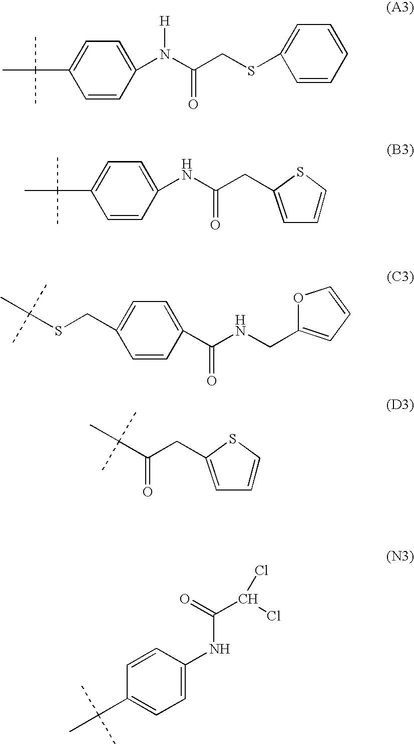

R₂ is selected, the same or each independently of the others, from the group comprising R₁, R₃ and/or a structural element (E3), (F3), (G3), (H3), (13), (J3), (K3), (L3), (M3), (O3) as indicated below:

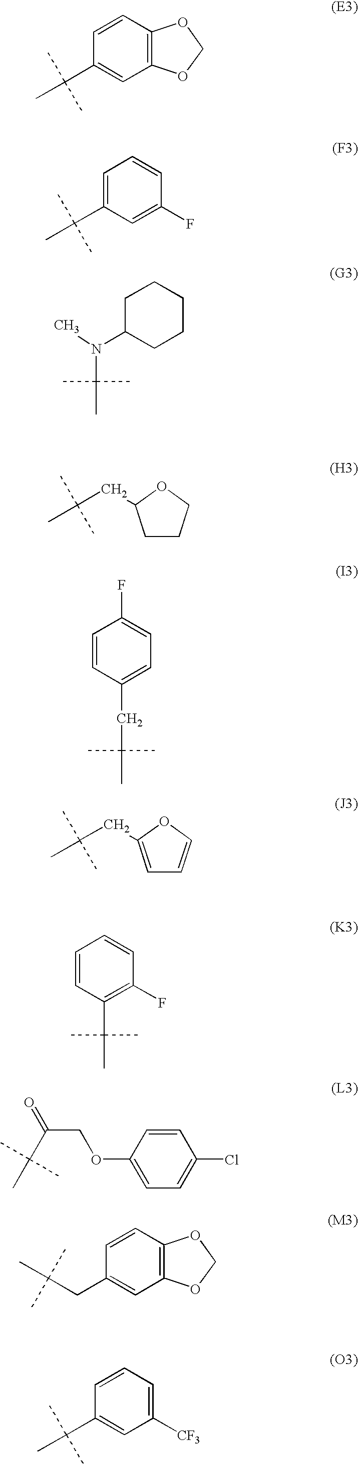

R₃ is selected, the same or each independently of the others, from the group comprising R₁, R₂ and/or selected from the group comprising hydrogen, OH, COOH, COO(C₁-C₁₀-alkyl), CONH₂, CONH(C₁-C₁₀-alkyl), CON(C₁-C₁₀-alkyl)₂, NHCO(C₁-C₁₀-alkyl), NHCOCHCl₂, halogen, preferably selected from the group comprising Cl, Br, F, CF₃, amine, C₁-C₁₀-alkyl and/or C₁-C₁₀-alkoxy.

Preferably, at least one or more of the structural elements R₁, R₂ and/or R₃ of the compounds (5), (6), (7) and (8) are selected, the same or each independently of the others, from the sulfurous structural elements (A3), (B3), (C3) and/or (D3). The compounds that can be used according to the invention can have several of the same and/or different structural elements (A3), (B3), (C3) and/or (D3). Preferably, the compounds that can be used according to the invention have at least one, preferably two, especially preferably three of the same and/or different structural elements (A3), (B3), (C3) and/or (D3). In other preferred embodiments, the compounds that can be used according to the invention have at least one structural element selected from the group comprising (E3), (F3), (G3), (H3), (I3), (J3), (K3), (L3), (M3) and/or (O3).

In preferred embodiments of the compounds that can be used according to the invention, at least one structural element R, R₁, R₂, R₃ and/or R₄, preferably at least one structural element R of the compounds (1), (2), (3) and (4), especially R₃ of the compounds (5), (6), (7) and (8), is preferably selected independently of the others from the group comprising C₁-C₅-alkyloxy, preferably selected from the group comprising —O-methyl, —O-ethyl, —O-isopropyl and/or —O-tert-butyl. Especially preferred among the C₁-C₅-alkoxy groups are methoxy- and/or ethoxy groups, and very especially preferred are methoxy groups.

It was surprisingly found that the compounds that can be used according to the invention have a significantly improved effect the smaller the alkoxy groups are. Thus it was surprisingly determined that a significant increase in efficacy in use of the compounds can be achieved when the alkoxy group R₃, especially of the compounds (5), is a C₁-C₂-alkoxy group, and a further increase in the efficacy of the compound can be achieved when the alkoxy group R₃ is a methoxy group.

In preferred embodiments, the compounds that can be used according to the invention are 1,2,4-triazoles selected from the group comprising compounds of the formulas (1) and/or (5). Preferably, R₁ of the compounds according to formula (5) is a structural element (A3), (B3), (C3), (D3) or (N3), R₂ is a structural element (E3), (F3), (G3), (H3), (I3), (J3), (K3), (L3), (M3) or (O3) and/or R₃ is a C₁-C₅-alkoxy group, preferably a methoxy or ethoxy group. Preferably, R₁ of the compounds according to formula (5) is a structural element (A3) or (B3), R₂ is a structural element (E3), (F3) or (K3) and/or R₃ is a methoxy or ethoxy group.

In very especially preferred embodiments, the compounds that can be used according to the invention are selected from the group comprising compounds (9), (10), (11), (22), (23) as indicated below, and/or the enantiomers, diastereomers, derivatives and pharmaceutically well-tolerated salts thereof:

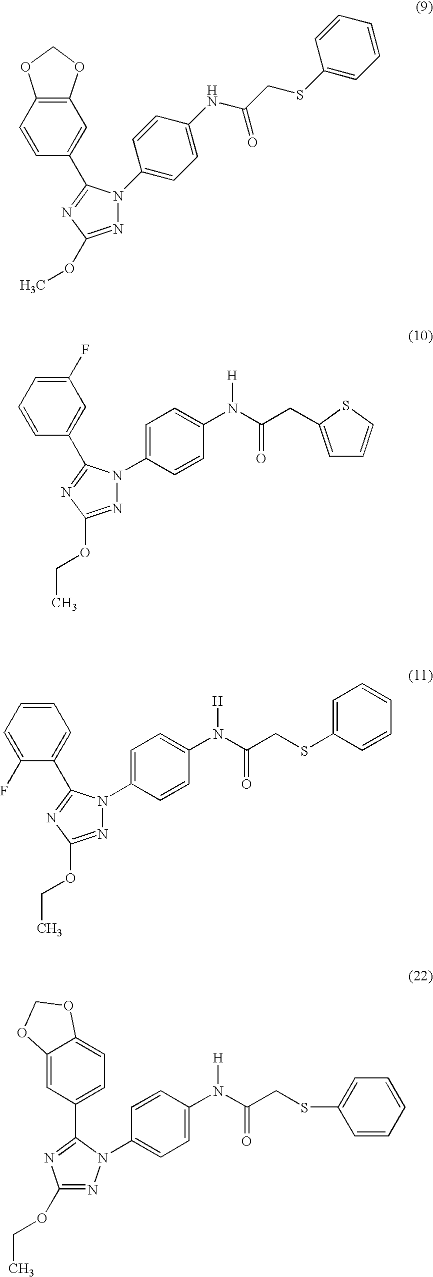

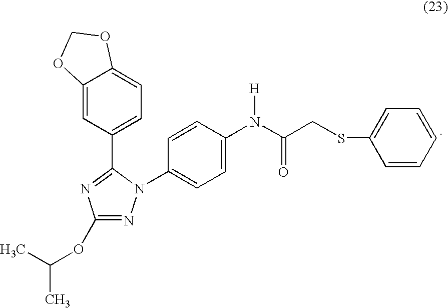

It was surprisingly found that especially 1,2,4-triazoles of the general formulas (1) and/or (5), especially according to formula (9), can produce an insulin resistance and/or can lead to chemically induced longevity.

In other preferred embodiments, the compounds that can be used according to the invention are selected from the group comprising compounds (12), (14), (20), (21) as indicated below and/or the enantiomers, diastereomers, derivatives and pharmaceutically well-tolerated salts thereof:

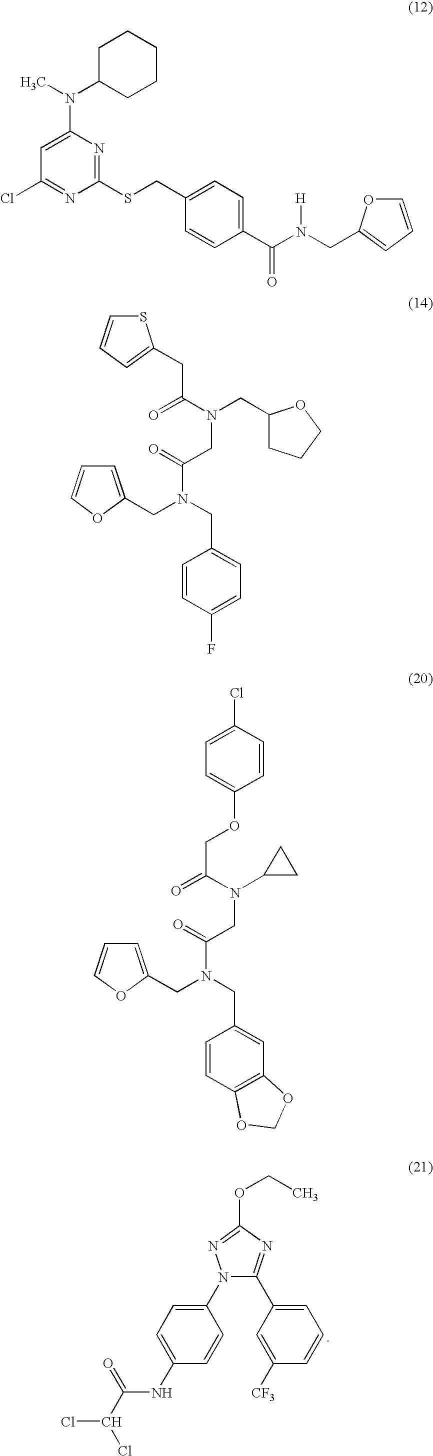

Another embodiment of the compounds that can be used according to the invention and/or the enantiomers, diastereomers and pharmaceutically well-tolerated salts thereof has the following formula (25):

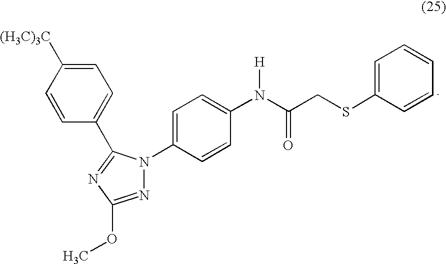

In further preferred embodiments, the compounds that can be used according to the invention can be derived, for example phosphorylated, glycolized, acetylated, ubiquitinylated, farnesylated, palmitoylated, geranylgeranylated and/or biotinylated.

Preferred derivatives are biotinylated compound [sic-Translator], with compound (24) as indicated below being especially preferred, for example, and/or the enantiomers, diastereomers, and pharmaceutically well-tolerated salts thereof

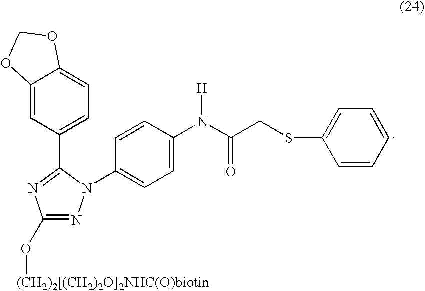

One advantage of the compounds that can be used according to the invention can be realized in that these compounds can exert an inhibitory effect on cytohesin-dependent signal cascades of the insulin and/or insulin-like growth factor (IGF) signaling pathway.

The compounds that can be used according to the invention can show, for instance in vivo in the mouse and the fly, that an insulin resistance can be induced. The compounds that can be used according to the invention can further show, in in-vitro experiments on human liver cells, that these can also develop an insulin resistance. An insulin resistance can lead to an increase in lifespan or longevity, opening up potential applications in the treatment of age-related illnesses.

One particular advantage of the compounds that can be used according to the invention can be provided in that these compounds can permit a chemically induced longevity within the framework of a therapeutic and/or preventive course of treatment. The term “chemically induced longevity” within the context of this invention means that by administering the compounds that can be used according to the invention, the lifespan of an organism and/or a tissue or organ can be extended. An extension of the lifespan of an organism and/or of a tissue or organ can advantageously be achieved by administering a compound that can be used according to the invention, without necessitating surgical treatment or a genetic alteration of the organism; in other words, longevity can be induced chemically especially by administering a substance.

The compounds that can be used according to the invention advantageously enable an influencing of the insulin and/or insulin-like growth factor (IGF) signaling pathway, and enable a use of the compounds that can be used according to the invention for the therapeutic and/or preventive treatment of diseases and pathological conditions that are linked to a regulation of the insulin and/or insulin-like growth factor (IGF) signaling pathway.

The regulation of the insulin and/or insulin-like growth factor (IGF) signaling pathway is influenced by a multitude of hormones and messenger substances, which are present in a complex equilibrium. Disrupted regulation can lead to a multitude of illnesses, such as obesity. Preferably, these are diseases and pathological conditions that are linked to the insulin and/or insulin-like growth factor (IGF) signaling pathway, especially diseases and pathological conditions caused by an increase in the insulin and/or insulin-like growth factor (IGF) signaling pathway. In preferred embodiments, the compounds that can be used according to the invention can effect an inhibition of the insulin and/or insulin-like growth factor (IGF) signaling pathway.

Preferably treatable diseases and pathological conditions that are linked to a regulation of the insulin and/or insulin-like growth factor (IGF) signaling pathway are selected from the group comprising obesity, cell aging, age-related cell damage, especially in the liver and/or the pancreas, age-related pathological conditions of liver and/or pancreatic cells, age-related functional disorders, such as a decreased regenerative power in the liver and/or the pancreas, cell stress, especially oxidative stress, especially stress induced by an increased metabolization of sugar, and/or apoptosis, especially β-cell apoptosis.

In particularly preferred embodiments, use of the compounds that can be used according to the invention can lead to an increased lifespan in animals, especially mammals, especially humans.

According to the invention, it has been found that the compound according to formula (9) can show a positive effect on lifespan in vivo. For instance, it was established through experimentation that the compound according to formula (9) was able in vivo to effectuate an increase in the lifespan of flies.

A chemically induced increase of lifespan or of age is a very particular advantage that can be provided by the compounds that can be used according to the invention.

Without being committed to a given theory, it is further assumed that the compounds that can be used according to the invention, especially the compound according to formula (9), can have a positive effect on the immune system. It is especially assumed that the compounds that can be used according to the invention, especially the compound according to formula (9), can induce an activation of the immune system.

Advantageously, the compounds that can be used according to the invention can have only a slight or negligible toxicity when administered. This enables their long-term use, for example. It also enables their administration for preventive purposes, especially in humans.

The compounds that can be used according to the invention can be administered using customary methods. Oral or parenteral administration is preferred, with oral administration being especially preferred. In preferred embodiments, the compounds that can be used according to the invention are formulated for oral or intravenous administration. Preferred inactive ingredients and/or solvents are selected from the group comprising DMSO (dimethylsulfoxide), glycerol and/or oil. Preferably, solvents are selected from the group comprising DMSO and/or vegetable oil, especially olive oil. One advantage of using oil, for example olive oil, is that this can provide an improvement in tolerance.

Examples that serve to illustrate the present invention are described in what follows.

### Material and Methods

### Cell Culture

Human hepG2 cells (ECACC) were cultivated in EMEM medium (Cambrex) with the addition of 10% fetal calf serum. 10⁵ cells were seeded in 2 ml medium in 6 well plates and were cultivated at 37° C. in a moist atmosphere with 5% CO₂ for 1 to 3 days, before being used for experiments.

### Mice

C57BL/6 mice (Charles River Laboratories) that had been kept in a pathogen-free animal facility, maintaining a 12-hour light/dark cycle, were used. The animals received standard mouse food (19% protein, 3.3% fat, 41.3% carbohydrates; ssniff Spezialdiäten GmbH) ad libitum. Male animals between the ages of 8 and 12 weeks were used.

### Flies

Drosophila flies (strain #6326 from the public Bloomington Stock Center, genotype: white¹¹¹⁸, URL http://flybase.org/.bin/fbidq.html?FBst0006326) were used; these were kept in groups of 20 flies, with a male to female ratio of 1:1, at 25° C. with standard fly food (0.53% (w/v) filamentous agar (“Gewürzmühle Brecht” company); 1.1% (w/v) brewer's yeast (“Gewürzmühle Brecht” company); 5.43% (w/v) cornmeal; 6.6% (v/v) sugar beet syrup (Grafschafter); 0.13% (w/v) Nipagin (methyl-4-hydroxybenzoate sodium salt, Merck company); 1.3% (v/v) ethanol (Roth company), 0.3% DMSO (Roth). For the experiments, adult flies zero to four hours after emerging from the pupa were used; the animals received new food every third day.

### EXAMPLE 1

### Investigation of the Transcription of IGFBP1 (Insulin-Like Growth Factor Binding Protein) with Regard to the Influencing of the Insulin Signaling Pathway in Liver Cells by the Compound According to Formula (9)

For human HepG2 cells (ECACC), serum was removed from the medium for 24 hours, the cells were incubated for an additional 12 hours with concentrations of 1.5625 μM, 3.125 μM, 6.25 μM, 12.5 μM, 25 μM or 50 μM of the compound according to formula (9) and with 10 nM insulin. Control cells received DMSO in a concentration corresponding to that of the treated cells.

Total RNA was isolated using the Absolutely RNA Kit (Stratagene), and the cDNA was produced by means of the reverse transcription of 2 μg RNA using the High Capacity cDNA Archive Kit (Applied Biosystems), according to the manufacturer's instructions. Quantitative PCR was performed in 10 μl batches in an iQ5 Cycler (BioRad) by means of the TaqMan Gene Expression Assay (Applied Biosystems) using gene-specific primer (Applied Biosystems), according to the manufacturer's instructions. The data were normalized to the β2-microglobulin expression.

It was determined that the compound according to formula (9) was able to nearly completely block the insulin-dependent inhibition of the transcription of IGFBP1 (insulin-like growth factor binding protein), a prototypical insulin-regulated gene. Thus in the presence of 12.5 μM of the compound according to formula (9) the expression, which was significantly reduced by 10 nM insulin, achieved nearly the value of the untreated cells.

### EXAMPLE 2

### Investigation of the Phosphorylation of Akt and FoxO1A with Regard to the Influencing of the Insulin Signaling Pathway in Liver Cells by the Compound According to Formula (9)

The expression of IGFBP1 (insulin-like growth factor binding protein) is controlled by insulin via the forkhead box transcription factors O1A (FoxO1A) and O3A (FoxO3A). Following insulin stimulation, the protein kinase B/Akt (PKB/Akt) is activated via phosphorylation, based upon the phosphoinositide-3-kinase (PI3K), reaches the nucleus where it phosphorylates the transcription factor O1A (FoxO1A), which leads to a decrease in the gene expression of IGFBP1, for example.

For human HepG2 cells (ECACC), serum was removed from the medium for 90 minutes, the cells were incubated for another 60 minutes with 5 μM, 10 μM and 15 μM of the compound according to formula (9), 200 nM Wortmannin or DMSO (dimethylsulfoxide) at a corresponding concentration, after which the cells were stimulated for 10 minutes with 100 nM insulin. The cells were then lysed in lysate buffer (20 mM Tris-Cl, pH 7.5, 150 mM NaCl, 1 mM EDTA, 1 mM EGTA, 2.5 mM sodium pyrophosphate, 1 mM β-glycerophosphate, 1 mM sodium vanadate, 1% Triton X-100) and protease inhibitor mix HP (Serva).

Standardized quantities of protein were separated out using SDS-PAGE, and were transferred to nitrocellulose membranes. For protein identification via immunoblotting, an antibody was used against the phosphorylated protein Akt, pAkt (Thr308) (cell signaling). Visualization was carried out using the Enhanced Chemiluminescence System (Millipore) using a VersaDoc 5000 CCD camera (BioRad), and the intensity of the bands was quantified using the QuantityOne software (BioRad).

It was determined that the compound according to formula (9) inhibited the insulin-dependent phosphorylation of both proteins Akt and FoxO1A, based upon concentration. Thus the application of 10 μM led to a 50-percent reduction in the phosphorylation of Akt as compared with phosphorylation following insulin stimulation, whereas 15 μM of the compound according to formula (9) led to a reduction in the phosphorylation of Akt to approximately 25% of the phosphorylation following insulin stimulation.

These examples demonstrate that the inhibition of cytohesins by the compound according to formula (9) in human liver cells leads to an inhibition of the insulin signaling pathway within the context of an insulin resistance.

### EXAMPLE 3

### Investigation Regarding the Generation of an Insulin Resistance In Vivo in the Mouse Using the Compound According to Formula (9)

A group of 6 C57BL/6 mice received standard mouse food mixed with 0.9 μmol/g of the compound according to formula (9) for a period of 3 days, while a control group of 6 animals received standard mouse food. The animals were then injected intraperitoneally with 100 μl normal saline solution (control group) or saline solution with 40 μg recombinant human insulin (Sigma). After 10 minutes the mice were anesthetized, the livers were removed and were lysed in lysate buffer (20 mM Tris-Cl, pH 7.5, 150 mM NaCl, 1 mM EDTA, 1 mM EGTA, 2.5 mM sodium pyrophosphate, 1 mM β-glycerophosphate, 1 mM sodium vanadate, 1% Triton X-100) and protease inhibitor mix HP (Serva).

Total RNA from 15 mg mouse liver in each case was isolated using the Absolutely RNA Kit (Stratagene) and the cDNA was produced via the reverse transcription of 2 μg RNA using the High Capacity cDNA Archive Kit (Applied Biosystems), according to the manufacturer's instructions. Quantitative PCR was performed in 10 μl batches in an iQ5-Cycler (BioRad) by means of the TaqMan Gene Expression Assay (Applied Biosystems) using primers (Applied Biosystems), against lgfbp1, Fbp2, Pck1, Pck2, G6 pc, Hk2, Pklr, Gck, Gckr, according to the manufacturer's instructions. The data were normalized to the β2-microglobulin expression. Studied were the expression of various genes that are controlled via the PI3K-dependent pathway of the insulin receptor signaling pathway, the FoxO-regulated model gene Igfbp1, pyruvate carboxykinase 1 and 2 (Pck1, Pck2), fructose-1,6-bisphosphatase 2 (Fbp2), and glucose-6-phosphatase (G6 pc), which participate in gluconeogenesis, and the glycolytic enzyme glucokinase (Gck), its regulators (Gckr), liver pyruvate kinase (Pklr) and hexokinase 2 (Hk2).

Further, standardized quantities of protein were separated out using SDS-PAGE, and this protein was identified via immunoblotting, after being transferred to nitrocellulose membranes, with an antibody, against the phosphorylated protein Akt, pAkt (Thr308) (cell signaling). Visualization and evaluation were performed as described in Example 2.

It was determined that the expression of the studied gluconeogenic gene, suppressed by insulin, was increased by the administration of the compound according to formula (9), whereas the expression of the glycolytic gene induced by insulin was decreased to a statistically significant degree. In concurrence with this result, it was also determined that the phosphorylation of the protein Akt stimulated by insulin was inhibited by approximately 40% by the administration of the compound according to formula (9).

This shows that the compound according to formula (9) in vivo produces a hepatic insulin resistance.

### EXAMPLE 4

### Investigation Regarding the Generation of an Insulin Resistance In Vivo in Flies Using the Compound According to Formula (9)

Three groups of 100 Drosophila larvae received 10 mg of the compound according to formula (9) mixed with 1 g autoclaved and pulverized brewer's yeast (Brewferm), while three control groups of 100 animals each received the uncut brewer's yeast. This food was administered on water-impregnated filters (Macherey-Nagel) for 3 days at 25° C.

In each case, more than 10 animals of the group were washed thoroughly with water, placed in lysate buffer (NucleoSpin RNA II kit, Macherey & Nagel), and homogenized for 1 minute at maximum speed (Ultra-Turrax T25basic). Total RNA was isolated using the NucleoSpin RNA II Kit (Macherey & Nagel, including DNase I-treatment on the column).

cDNA was produced from each 500 ng total RNA using the QuantiTect Reverse Transcription Kit (Qiagen), according to the manufacturer's instructions, including DNase I treatment. PCR was performed in batches with a total volume of 25 μl (iQ5 Real Time PCR Detection System, BioRad). The batches contained 1 μl of the cDNA batch, in each case 200 nM and 3′- and 5′-primer (Metabion) 12.5 μl 2×iQ5 SYBR Green Supermix (BioRad).

The PCR program that was used comprised 40 cycles with the following steps: 15 seconds denaturing at 95° C., 30 seconds annealing at 59° C. and 30 seconds extension at 72° C. Evaluation was carried out using the iQ5 Optical System software (Version 1.1.1442.OCR, BioRad). Actin 5C (Act5C, act) and the ribosomal protein L32 (RpL32, rp49) were used as reference genes.

It was determined that the expression of the studied insulin-repressed genes 1nR (insulin receptor) and 4E-BP, a translation repressor, was increased more than four times in the case of 4E-BP and more than double in the case of 1nR by the administration of the compound according to formula (9).

### EXAMPLE 5

Investigation of the Phosphorylation of Akt by the Compound According to Formula (9) in the Drosophila S2 Schneider Cell Line

10⁶ Drosophila S2 cells (Schneider, 1972. J. Embryol. Exp. Morphol. 27, 353) were seeded in cell culture medium (PAN company) with 10% fetal calf serum and 1% streptomycin, penicillin (Gibco) in 35 mm cell culture plates (Nunc company) and cultivated for 2 days at 25° C. to 70-80% confluence. After 2 days, 0.5% (v/v) of the medium DMSO was added, and, based upon the experiment, 10 μM of the compound according to formula (9) was added for 2 hours before insulin stimulation. For an insulin stimulation, human insulin (Sigma) was used for 15 minutes at a rate of 10 μg/ml. Once stimulation was complete, the cells were washed with cold PBS, lysed in cold (4° C.) lysate buffer (50 mM Tris-HCl, pH 8.0, 150 mM NaCl, 1.0% Triton X-100, 0.1% SDS, 0.5% sodium deoxycholate, 50 mM NaF, 100 μM sodium orthovanadate, 1 mM phenylmethylsulfonyl fluoride and EDTA-free protease inhibitor (Roche), according to manufacturer's instructions, and used in the Western Blot analysis.

In each case, 10 μg protein were isolated on a 12% SDS polyacrylamide gel and transferred to 0.22 μm porous nitrocellulose membranes (Amersham). The membranes were blocked using 5% (w/v) milk powder (Roth company) in TBST (150 mM NaCl; 50 mM Tris pH 7.4; 0.1% (v/v) Tween-20). Incubation with the primary antibody was carried out overnight in 5% (w/v) BSA in TBST. The primary antibodies Anti-Phospho Akt (Ser505) and Anti-Akt (cell signaling) were each used in a dilution of 1:1000. Incubation with the secondary antibody was carried out using peroxidase-coupled goat-anti-rabbit immunoglobulin (Santa Cruz, serum ID sc-2004) or peroxidase-coupled donkey-anti-mouse immunoglobulin (Santa Cruz, serum ID sc-2314) in a dilution of 1:15000.

Visualization was performed by means of the ECL Kit (Enhanced Chemiluminescence, Amersham) using the VersaDoc 5000 Imaging System (BioRad) and the QuantityOne software (BioRad).

In concurrence with the results of Example 4, it was determined that the insulin-stimulated phosphorylation of the protein Akt was decreased by half by the administration of the compound according to formula (9).

This shows that the compound according to formula (9) induces insulin resistance in vivo in the fly, which can result in an increase in longevity.

### EXAMPLE 6

### Investigation of the Influence of the Insulin Signaling Pathway by the Compound According to Formula (9) and the Compound According to Formula (25) In Vitro in Cells of the Drosophila S2 Schneider Cell Line

Drosophila Schneider 2 (S2) cells were cultivated in Schneider's Medium (PAN company) with 10% heat-inactivated fetal calf serum (FCS). 2×10⁶ cells were drawn into 35 mm vessels at 25° C. to 80% confluence, washed one time in phosphate buffered saline solution (1×PBS, Gibco), and transferred for twelve hours to Schneider's Medium, without FCS. The cells were then preincubated for two hours with 0.5% DMSO containing Schneider's Medium, without FCS, with or without the compound according to formula (9) or formula (25), with the final concentrations of the compounds according to formulas (9) and (25) being 1 μM, 10 μM and 100 μM. This was followed by a four-hour stimulation with 10 μg/ml human insulin (Sigma).

After washing the cells two times with PBS, total RNA was then isolated from the cells using the NucleoSpin RNA II Kit (Macherey & Nagel), according to the manufacturer's instructions. The first strain cDNA synthesis was performed with every 500 ng RNA, using the QuantiTect Reverse Transcription Kit (Qiagen), according to the manufacturer's instructions. The real-time PCR reaction was carried out using the SYBR Green Supermix (BIO-RAD), according to the manufacturer's instructions, in the iQ5 Real-Time PCR Detection System (BIO-RAD). In this process, PCR was performed in batches having a total volume of 25 μl, with each batch containing 1 μl of the cDNA batch, 200 nM 3′ and 5′ primer (Metabion) and 12.5 μl 2×iQ5 SYBR Green Supermix (BioRad). The PCR program that was used comprised 40 cycles with the following steps: 15 seconds denaturing at 95° C., 30 seconds annealing at 59° C. and 30 seconds extension at 72° C. Evaluation of the real time data was performed according to manufacturer's instructions using the BIO-RAD iQ5 Optical System software (Version 1.1.1442.OCR).

The activity of the insulin signaling pathway was determined from the transcription rate of the insulin target gene Drosophila eukaryotic initiation factor 4E binding protein (d4EBP, Thor). The genes Actin 5C (Act5C, act) and ribosomal protein L3 (RpL32, rp49) were used as reference genes. Table 1 shows the sequences of the oligonucleotides used for the real time analysis.

TABLE 1
Oligonucleotide Sequences

| | Forward Primer | Reverse Primer |
|---|---|---|
| Gene | Sequence (Metabion) | Sequence (Metabion) |
| Act5C | GTGCACCGCAAGTGCTTCTAA | TGCTGCACTCCAAACTTCCAC |
| | (SEQ. ID NO: 1) | (SEQ. ID NO: 2 |
| RpL32 | GCTAAGCTGTCGCACAAATG | GTTCGATCCGTAACCGATGT |
| | (SEQ. ID NO: 3) | (SEQ. ID NO: 4) |
| d4EBP | CATGCAGCAACTGCCAAATC | CCGAGAGAACAAACAAGGTGG |
| | (SEQ. ID NO: 5) | (SEQ. ID NO: 6) |

It was determined that in Drosophila Schneider 2 (S2) cells that were treated with insulin, d4EBP was transcriptionally inhibited to less then 35% of the transcription rate of the control groups. This inhibitory effect was suppressed by the compounds according to formulas (9) and (25) at a concentration of 100 μM, wherein the transcription rate of d4EBP increased to more than 85% of that of the untreated control groups as a result of incubation with 100 μM of the compound according to formula (9), and to more than 95% of that of the untreated control groups as a result of incubation with 100 μM of the compound according to formula (25).

It was therefore demonstrated that the two compounds according to formulas (9) and (25) influenced the insulin signaling pathway at a concentration of 100 μM and in vitro led to an insulin resistance.

### EXAMPLE 7

### Investigation of the Influence on the Lifespan of Adult Drosophila melanogaster Flies by the Compounds According to Formulas (9) and (25)

### Flies

The experiments were conducted on isogenic flies (Drosophila melanogaster) of the genotype white¹¹¹⁸, also called white⁻ or w⁻, and mutants (w¹¹¹⁸; step^{k08110}/step^{SH0323}) (strain # 10770 from the public Bloomington Stock Center, genotype: step^{k08110}/CyO, URL http://flybase.bio.indiana.edu/reports/FBstOO10770.html; strain # FBst0103734 from the public Szeged Stock Center, genotype: step^{SH0323}/CyO, URL http://flybase.bio.indiana.edu/reports/FBst0103734.html), in which the Drosophila cytohesin steppke is mutated and therefore defective.

By crossing the two steppke alleles k08110 and SH0323 eight times with whites flies, an isogenic genetic background is produced, which permits a comparison of the lifespans between the genotypes of the type white¹¹¹⁸ and the mutants (w¹¹¹⁸; step^{k08110}/step^{SH0323}). The average lifespan of the wild type was approximately 25 days, while that of the mutants (w¹¹¹⁸; step^{k08110}/step^{SH0323}) was approximately 30 days.

### Special Food

To produce the special food, 32.5 g autolysed yeast (Fluka (Catalog No. 73145)) and 10 g agar (Difco (Catalog No. 281230)) were mixed with 300 ml water (demineralized, Roth (Catalog No. 3175)) and autoclaved at 120° C. for 20 minutes. The solution was cooled to 60° C., and 32.5 g α-D(+)-glucose monohydrate (Roth (Catalog No. 6887)) were added under stirring. 15 ml 10% (w/v) p-hydroxybenzoic acid methylester (Sigma (Catalog No. H3647)) solution dissolved in 70% (v/v) ethanol (Roth (Catalog No. 9065)) and 1.35 ml dimethylsulfoxide (DMSO, Roth (Catalog No. A994)) were then added. Water was then added to this basic solution to a volume of 450 ml, which was then held at a temperature of 60° C. For the control food, 50 ml of a 0.7% (v/v) DMSO solution in water were produced. For the food containing the compounds according to formulas (9) or (25), 0.2 ml of a 25 mM solution of the compounds according to formula (9) or (25) in 100% DMSO were added to 49.8 ml of a 0.3012% DMSO solution in water.

These 50 ml of the control solutions and the solutions containing the compounds according to formula (9) or (25) were each mixed with 450 ml of the basic solution, and were placed in 4 ml amounts in polystyrene containers (height 9.5 cm, diameter 2.4 cm, sealed with cotton wadding) to maintain the flies. After 24 hours, the cooled containers were sealed with cotton wadding and stored at 4° C.

The final concentration of the food components in the control food was 6.5% (w/v) autolysed yeast, 6.5% (w/v) α-D(+)-glucose monohydrate, 2% (w/v) agar, 0.3% (w/v) p-hydroxybenzoic acid methylester, 2.1% ethanol (v/v), 0.34% (v/v) DMSO.

The final concentration of the food components in the food containing the compound according to formula (9) was 6.5% (w/v) autolysed yeast, 6.5% (w/v) α-D(+)-glucose monohydrate, 2% (w/v) agar, 0.3% (w/v) p-hydroxybenzoic acid methylester, 2.1% ethanol (v/v), 0.34% (v/v) DMSO, 10 μM compound according to formula (9).

The final concentration of the food components in the food containing the compound according to formula (25) was 6.5% (w/v) autolysed yeast, 6.5% (w/v) α-D(+)-glucose monohydrate, 2% (w/v) agar, 0.3% (w/v) p-hydroxybenzoic acid methylester, 2.1% ethanol (v/v), 0.34% (v/v) DMSO, 10 μM compound according to formula (25).

### Fly Maintenance

The larvae were maintained on standard fly food containing 1.1% (w/v) brewer's yeast (“Gewürzmühle Brecht” company), 5.43% (w/v) cornmeal, 0.53% (w/v) filamentous agar (“Gewürzmühle Brecht” company), 6.6% (v/v) sugar beet syrup (Grafschafter), 1.3% (v/v) ethanol (Roth company), 0.13% (w/v) p-hydroxybenzoic acid methylester (Sigma).

24 hours after emergence of the imagines, the adult flies, females and males were separated under a brief CO₂ anesthetization lasting less than 2 minutes. During the 24 hours after emergence, the animals were fed using standard fly food. Females and males were then separated and the females were kept in 10 groups of 20 animals each in polystyrene containers, height 9.5 cm, diameter 2.3 cm, sealed with cotton wadding, using 4 ml special food with or without the compounds according to formulas (9) or (25). The special food was freshly produced every second week.

The flies were switched to fresh special food every second or third day, and the dead animals were counted. Analysis of lifespan was performed using the Kaplan-Meier Analysis, with the help of statistical software (XLSTAT).

The Kaplan-Meier analysis showed a significant increase in average lifespan both for the control animals of the wild type w¹¹¹⁸ and for the mutants (w¹¹¹⁸; step^{k08110}/step^{SH0323}) when they were fed with the compounds according to formula (9), as shown in Table 2.

TABLE 2
Genotypes, Treatment and Lifespans

| | | Number | Average Lifespan |
|---|---|---|---|
| Genotype | Treatment | N | [Days] |
| w¹¹¹⁸ | — | 201 | 25.2 (±0.9) |
| w¹¹¹⁸; step^{k08110}/step^{SH0323} | — | 194 | 30.0 (±0.8)‡ |
| w¹¹¹⁸ | 10 μM | 202 | 28.1 (±1.0)* |
| | Compound (9) | | |
| w¹¹¹⁸; step^{k08110}/step^{SH0323} | 10 μM | 196 | 34.6 (±0.8)† |
| | Compound (9) | | |
| w¹¹¹⁸ | 10 μM | 208 | 25.0 (±0.7) |
| | Compound (25) | | |
| w¹¹¹⁸; step^{k08110}/step^{SH0323} | 10 μM | 200 | 29.3 (±0.7) |
| | Compound (25) | | |

The values show the average lifespan in days (±margin of error). The lifespan and significance were calculated using the Kaplan Meier Analysis with the help of the XLSTAT Life software, wherein:
‡P < 0.001 against w1118 (without treatment).
*P < 0.05 against w1118 (without treatment).
†P < 0.001 against w1118; stepk08110/stepSH0323 (no treatment).

Control animals that were fed with the compounds according to formula (9) had a significantly increased average lifespan (P<0.05) of 11.5% as compared with the untreated control animals. Whereas the mutants lived 19% longer than the control animals (significance P<0.001), feeding with the compounds according to formula (9) led to a greater increase in the average lifespan of 15.3% (significance P<0.001), so that these mutants had a 37.3% longer lifespan than untreated control animals.

This shows that an administration of the compounds according to formula (9) can lead to a significant increase in lifespan in flies.

A further increase in lifespan could be achieved in flies in which the Drosophila cytohesin steppke was mutated and the protein quantity of the Drosophila cytohesin steppke was reduced.

This shows that the compound according to formula (9) is capable of producing a greater increase in lifespan. Especially, this shows that the increase in the lifespan of the mutant flies can be attributed to an inhibition of the fly cytohesin steppke.

Conversely, with the compounds according to formula (25), no significant change in lifespan could be achieved either in the control animals or in the mutants.

### EXAMPLE 8

### Measurement of Food Intake of Flies

In order to demonstrate that the increased lifespan, shown in Example 7, of the flies fed with the compound according to formula (9) could not be attributed to the fact that this experimental group ingested less food due to the taste or smell of the compound according to formula (9), and therefore lived longer, the food intake of flies was measured using the Capillary Feeder Assay (CAFÉ), and was statistically evaluated.

The procedure described in Ja et al. (Ja et al., Prandiology of Drosophila and the CAFÉ assay. PNAS 2007 May 15; 104(20):8253-6) was used. Every five groups of two female adult flies each were exposed to the various feeding conditions with or without the compound according to formula (9), over a period of ten days. The measurement of the food intake per hour and per fly was performed on days 1, 2, 3, 5, 6, 7, 8, 9 and 10 of the ten days.

The control food contained 5% (w/v) autolysed yeast, 5% (w/v) α-D(+)-glucose monohydrate, 0.3% (w/v) p-hydroxybenzoic acid methylester, 2.1% ethanol (v/v), 0.34% (v/v) DMSO. The food with the compound according to formula (9) contained 5% (w/v) autolysed yeast, 5% (w/v) α-D(+)-glucose monohydrate, 0.3% (w/v) p-hydroxybenzoic acid methylester, 2.1% ethanol (v/v), 0.34% (v/v) DMSO, 10 μM compound according to formula (9).

It was determined that the animals fed with the compound according to formula (9) did not at any time eat significantly less than the control food animals. Thus the established increase in lifespan resulting from the compound according to formula (9) cannot be attributed to a decreased food intake.

1-11. (canceled)

12: A process for the therapeutic and/or preventive treatment of diseases and pathological conditions in a subject that are linked to a regulation of the insulin and/or insulin-like growth factor (IGF) signaling pathway and/or for chemically inducing longevity, the process comprising administering to the subject a pharmaceutical preparation comprising one or more compounds selected from the group consisting of the formulas (1), (2), (3) and (4): 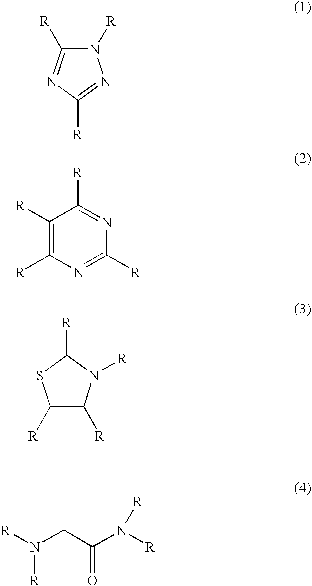
wherein:
R is selected, the same or each independently of the others, from the group consisting of hydrogen, OH, COOH, COO(C₁-C₁₀-alkyl), CONH₂, CONH(C₁-C₁₀-alkyl), CON(C₁-C₁₀-alkyl)₂, NHCO(C₁-C₁₀-alkyl), NHCOCHCl₂, halogens, CF₃, amine, C₁-C₁₀-alkyl, C₁-C₁₀-alkoxy and a structural element according to the formula (A1), (B1), (C1), (D1), (E1), (F1), (G1), (H1), (I1), (J1), (L1), (M1): 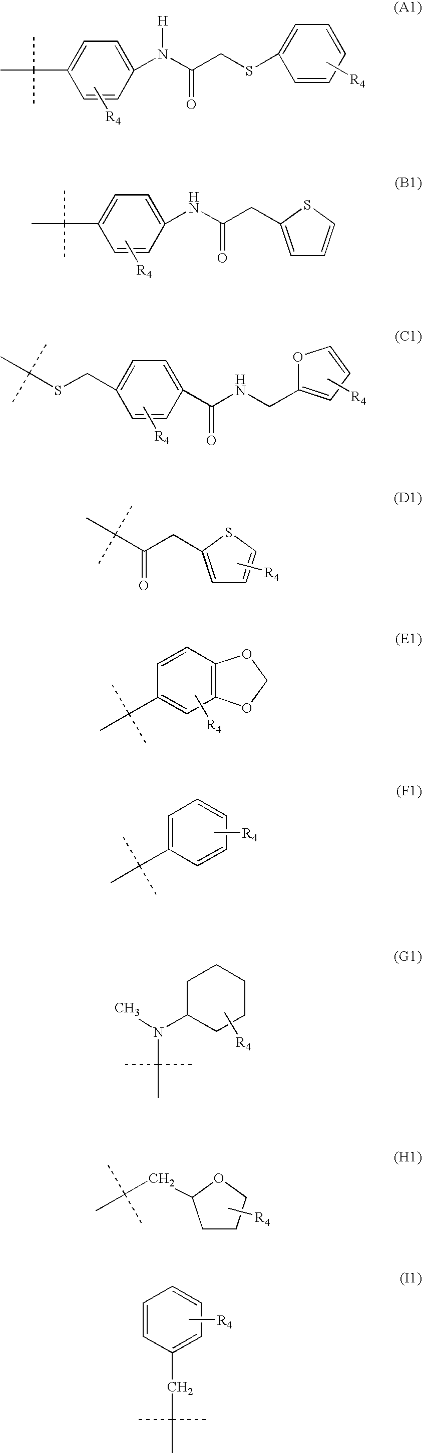 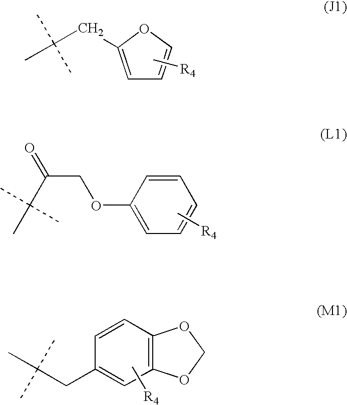
wherein:
R₄ is selected, the same or each independently of the others, from the group consisting of hydrogen, OH, COOH, COO(C₁-C₁₀-alkyl), CONH₂, CONH(C₁-C₁₀-alkyl), CON(C₁-C₁₀-alkyl)₂, NHCO(C₁-C₁₀-alkyl), NHCOCHCl₂, halogens, CF₃, amine, C₁-C₁₀-alkyl and/or C₁-C₁₀-alkoxy,
and/or the enantiomers, diastereomers, derivatives and pharmaceutically well-tolerated salts thereof.

13: The process according to claim 12 wherein the halogens for R are selected from the group consisting of Cl, Br and F.

14: The process according to claim 12 wherein the halogens for R₄ are selected from the group consisting of Cl, Br and F.

15: The process according to claim 12 wherein the one or more compounds are selected from the group consisting of compounds according to the formulas (1), (2), (3) and (4) and compounds according to the formulas (5), (6), (7) and (8): 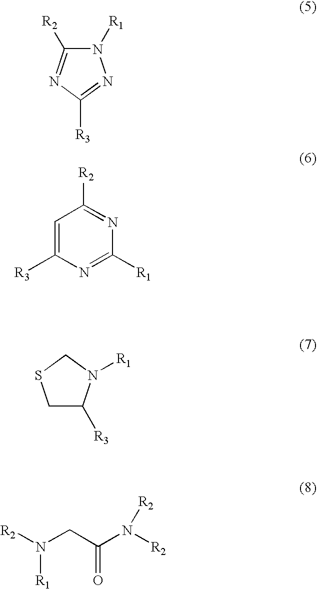
wherein:
R_{1′} is selected, the same or each independently of the others, from the group consisting of R₂, R₃ and a structural element according to the formula (A2), (B2), (C2), (D2), (N2): 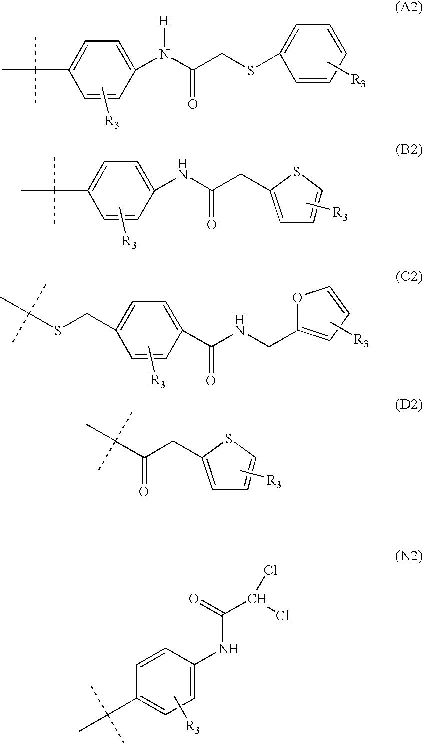
R₂ is selected, the same or each independently of the others, from the group consisting of R₁, R₃ and a structural element according to the formula (E2), (F2), (G2), (H2), (I2), (J2), (K2), (L2), (M2), (O2): 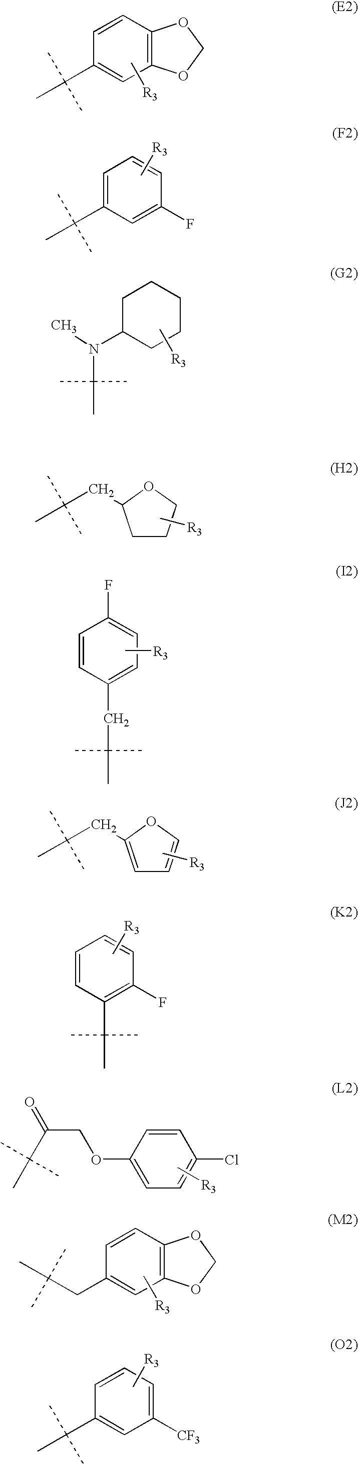
R₃ is selected, the same or each independently of the others, from the group consisting of R₁, R₂, hydrogen, OH, COOH, COO(C₁-C₁₀-alkyl), CONH₂, CONH(C₁-C₁₀-alkyl), CON(C₁-C₁₀-alkyl)₂, NHCO(C₁-C₁₀-alkyl), NHCOCHCl₂, halogens, CF₃, amine, C₁-C₁₀-alkyl and/or C₁-C₁₀-alkoxy,
and/or the enantiomers, diastereomers, derivatives and pharmaceutically well-tolerated salts thereof.

16: The process according to claim 15 wherein the halogens for R₃ are selected from the group consisting of Cl, Br and F.

17: The process according to claim 12 wherein the one or more compounds are selected from the group consisting of compounds according to the formulas (5), (6), (7) and (8): 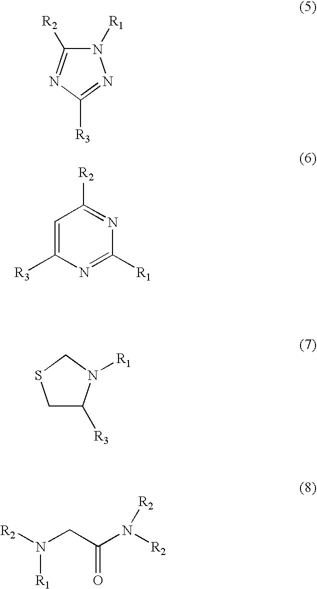
wherein:
R₁ is selected, the same or each independently of the others, from the group consisting of R₂, R₃ and a structural element according to the formula (A3), (B3), (C3), (D3), (N3): 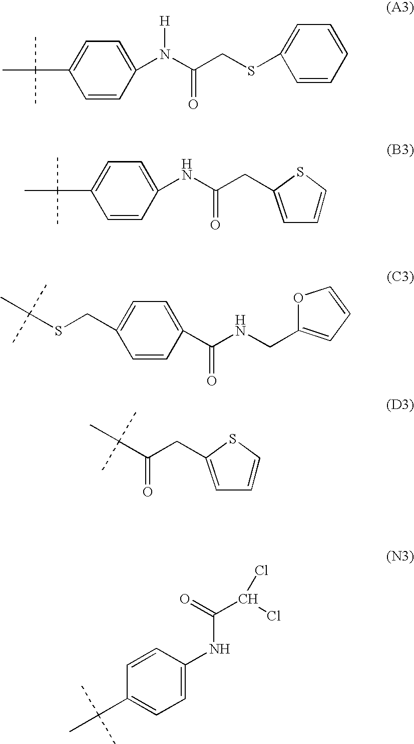
R₂ is selected, the same or each independently of the others, from the group consisting of R₁, R₃ and a structural element according to the formula (E3), (F3), (G3), (H3), (I3), (J3), (K3), (L3), (M3), (O3): 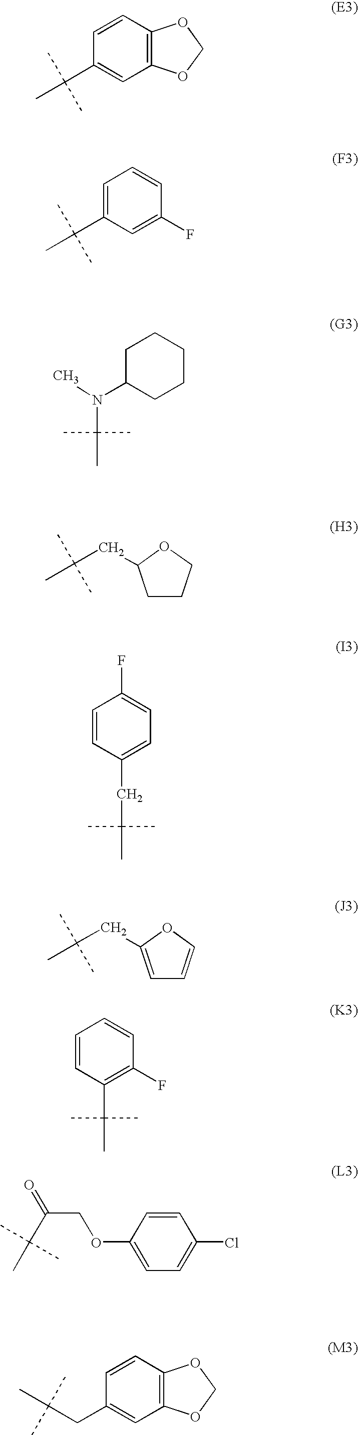 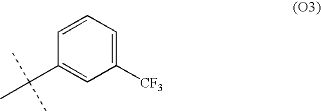
R₅ is selected, the same or each independently of the others, from the group consisting of R₁, R₂, hydrogen, OH, COOH, COO(C₁-C₁₀-alkyl), CONH₂, CONH(C₁-C₁₀-alkyl), CON(C₁-C₁₀-alkyl)₂, NHCO(C₁-C₁₀-alkyl), NHCOCHCl₂, halogens, CF₃, amine, C₁-C₁₀-alkyl and/or C₁-C₁₀-alkoxy,
and/or the enantiomers, diastereomers, derivatives and pharmaceutically well-tolerated salts thereof.

18: The process according to claim 17 wherein the halogens for R₃ are selected from the group consisting of Cl, Br and F.

19: The process according to claim 15 wherein R, R₁, R₂, R₃ and/or R₄ is a C₁-C₅-alkoxy group.

20: The process according to claim 19 wherein the C₁-C₅-alkoxy group is selected from the group consisting of —O-methyl, —O-ethyl, —O-isopropyl and —O-tert-butyl.

21: The process according to claim 12 wherein the one or more compounds are selected from the group consisting of compounds according to the formulas (9), (10), (11), (22), (23) as indicated below, and/or the enantiomers, diastereomers and pharmaceutically well-tolerated salts thereof:

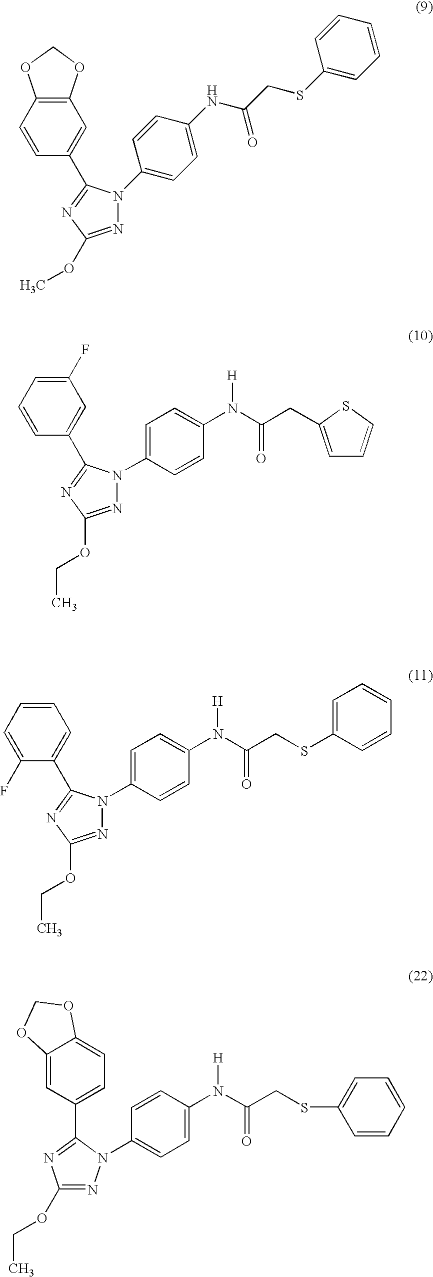

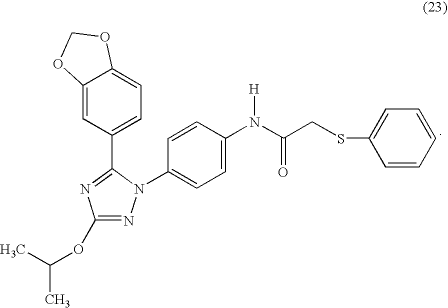

22: The process according to claim 12 wherein the one or more compounds are selected from the group consisting of compounds according to the formulas (12), (14), (20), (21) as indicated below, and/or the enantiomers, diastereomers and pharmaceutically well-tolerated salts thereof:

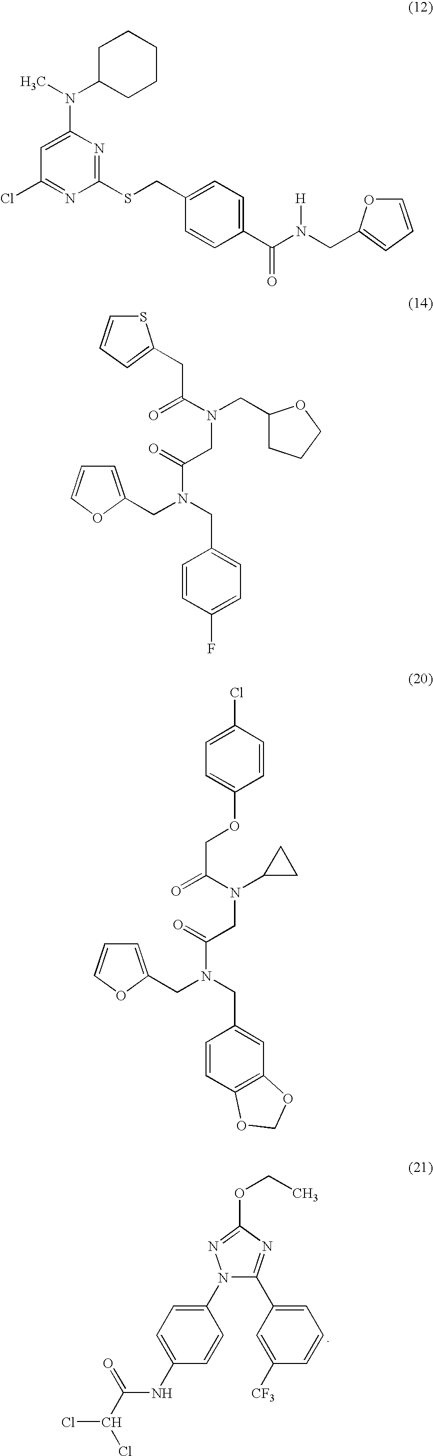

23: The process according to claim 12 wherein the derivative is a biotinylated compound and/or the enantiomers, diastereomers and pharmaceutically well-tolerated salts thereof.

24: The process according to claim 23 wherein the biotinylated compound is a compound according to formula (24) as indicated below, and/or the enantiomers, diastereomers and pharmaceutically well-tolerated salts thereof:

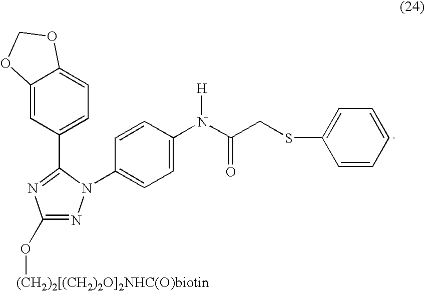

25: The process according to claim 12 wherein the diseases and pathological conditions that are linked to a regulation of the insulin and/or IGF signaling pathway are selected from the group consisting of obesity, cell aging, age-related cell damage, age-related pathological conditions of liver and/or pancreatic cells, age-related functional disorders in the liver and/or pancreas, cell stress and apoptosis.

26: The process according to claim 12 wherein the diseases and pathological conditions that are linked to a regulation of the insulin and/or IGF signaling pathway are selected from the group consisting of age-related cell damage in the liver and/or the pancreas, oxidative cell stress induced as a result of increased sugar metabolism and β-cell apoptosis.

27: The process according to claim 12 wherein the subject is a mammal and the administration of the pharmaceutical composition increases the life span of the mammal.

28: The process according to claim 12 wherein the pharmaceutical preparation is formulated for oral or intravenous administration.

29: The process according to claim 12 wherein the pharmaceutical preparation is present in a solvent selected from the group consisting of DMSO, glycerol and vegetable oil.

30: The process according to claim 12 wherein the pharmaceutical preparation is present in a solvent selected from the group consisting of DMSO and vegetable oil.

31: The process according to claim 30 wherein the vegetable oil is olive oil.


SEQUENCE LISTING (6 sequences)

SEQ ID NO: 1 - DNA, 21 bp, Artificial
  Forward Primer
gtgcaccgcaagtgcttctaa

SEQ ID NO: 2 - DNA, 21 bp, Artificial
  Backward Primer
tgctgcactccaaacttccac

SEQ ID NO: 3 - DNA, 20 bp, Artificial
  Forward Primer
gctaagctgtcgcacaaatg

SEQ ID NO: 4 - DNA, 19 bp, Artificial
  Backward Primer
gttcgatccgtaaccgatg

SEQ ID NO: 5 - DNA, 20 bp, Artificial
  Forward Primer
catgcagcaactgccaaatc

SEQ ID NO: 6 - DNA, 21 bp, Artificial
  Backward Primer
ccgagagaacaaacaaggtgg